(19) United States
(12) Patent Application Publication
(10) Pub. No.: US 2005/0142231 A1
(43) Pub. Date: Jun. 30, 2005

(54) Pharmaceutical activity and manufacturing method of the five-plant compound hypolipemic products

(76) Inventors: Jiao Gong, Arcadia, CA (US); Wei Jiang, Arcadia, CA (US)

Correspondence Address: James Chen, #560, 150 N Santa Anita Ave., Arcadia, CA, 91006, US

(21) Appl. No.: 10/745,798

(22) Filed: Dec. 29, 2003

(51) Int. Cl. A61K 35/78

(52) U.S. Cl. 424/762; 424/773; 424/777

(57) ABSTRACT

World Health Organization thinks that the rise in triglyceride is a major cause for occurrence of atherosclerosis. The U.S. classical anti-arteriosclerosis drug—atromid-s can reduce TG and VLDL remarkably and inhibit the synthesis and release of TG the effects of the five-plant compound hypolipemic products are better than that of atromid-s. They are made under delicate and strictly controlled conditions by absorbing, eluting, isolating and purifying the five-plant components with supercritical CO₂ extraction and WLD resin absorption techniques and quantitative analysis by fingerprinting through high performance liquid chromatography to ensure the uniformity and stability of product quality. The results of experiments on pharmacological activity show the compound products have the pharmacological action to inhibit the rise in serum cholesterol in rabbit, enhance LCAT activity and reduce the content of serum cholesterol and triglyceride, clinically They can be used in the treatment of atherosclerosis and obesity.

### BACKGROUND OF THE INVENTION

The pharmacological activity and manufacturing process of the five-plant compound hypolipermic products, belong to the combined patent on the pharmacological activity of natural products and methods of isolating and purifying the same products in the U.S. patents. The five-plant compound products have the pharmacological activity to inhibit the rise in serum cholesterol in rabbit remarkably, reduce the content of serum cholesterol and triglyceride and enhance the activity of the esterificartion of cholesterol noticeably.

In the U.S.A., patent protection is provided for not only the drugs used in the treatment of diseases, but also the curative methods. If an invention is claimed to have the practicability of curing diseases in people or animals, then the applicable standard to judge whether the claimed practicability meets the requirement of the practicability specified in the Patent Law should be the same as that in other technical fields. It is specially stressed that the requirement of practicability of treatment in the Patent Law should not be confused with that of safety and efficacy for drugs sold in the U.S. market specified by FDA of the U.S. The basis on which the practicability of the invention is assessed in the action of the treatment, prevention of disease or pharmacological activity of the invention. The actual requirement of practicability is satisfied by identifying the invention of the compound with pharmacological activity. The pharmacological activity and manufacturing process of the five-plant compound hypolipemic products have met the requirements of patentability and patent practicability by U.S. Trademark & Patent Administration.

Atheroslerosis is an affection occurring in the wall of the artery with lipid deposition, cell infiltration, cell proliferation in vascular smooth muscle, increased extracellular stromata including collagen and elastin as well as formation of foamy cell in some parts of the body. As a result the endarterial membrane is thickened, deep atheroma is softned and vascular cavity blocked, even disrupted. This is the main pathological foundation of many cardiovascular diseases.

Lipometabolism disorder is a major factor for formation of AS. The lipids in the plasma cover total cholesterol (TC), triglyceride(TG), phospholipid and free fatty acid; TC includes cholesterol(CH) and cholesterol ester (CE): They are combined with different apolipoproteins and lipoproterins in different proportion in the plasma. The lipoproterins can be classified into chylomicron (CM), very low density lipoproterin (VLDL), low density lipoprotein (LDL), high density lipoprotein (HDL) and apolipoprotein (a). CM is mainly for carrying exogenous TG; VLDL is finally decomposed to LDL; LDL contains rich CH; which is the chief component for the formation of AS. HDL carries CH in the wall of the artery to the kidney for metabolism through antiport and is therefore resistant to AS.

At present, CH content is still the indicator in the clinical determination of various lipoproteins, such as LDL-Cholesterol (LDL-C), VLDL-Cholesterol (VLDL-C) and HDL-Cholesterol (HDL-C). Unusual rise in TC, TG, LDL or VLDL is called hyperlipemia, which is classified into the types shown in the following table by WHO.

Classification of Hyperlipemia

| Type | I | IIa | IIb | III | IV | V |
|---|---|---|---|---|---|---|
| Rise | CM | LDL | VLDL | B-VLDL | VLDL | CM |
| Lipoprotein | | | LDL | | | VLDL |
| Serum lipid | | TC+++ | TC++ | TC++ | TC+ | TC+ |
| | TG+++ | | TG++ | TG++ | TG++ | TG+++ |

Note:
+rise

Besides, unusual fall in HDL and rise in LP (a) are also major contributory factors to AS. The classical hypolipemic is atromid-S, which can reduce TG and VLDL markedly, reduce TC and LDL-C mildly, reduce HDL-C mildly, increase the activity of LDL, control synthesis and release of TQ strengthen the elimination of LDL and promote synthesis of apo A. The compound products have a more pronounced effect to enhance the esterification of cholesterol than atromid-S.

### BRIEF SUMMARY OF THE INVENTION

Rabbits were fed with high cholesterol and lipid feed. In several weeks, remarkable hyperlipemia occurred. The compound products can reduce the source of serum cholesterol by inhibiting part of exogenous cholesterol absorption and suppressing synthesis of endogenous cholesterol, and promote the removal and re-distribution of cholesterol tissue by increasing the conversion and excretion of cholesterol so as to speed up the consumption of cholesterol, thereby inhibiting the rise in serum cholesterol. The results of pharmacological activity experiment show that the compound products can remarkably inhibit the rise in cholesterol in rabbits fed with feed containing cholesterol and high lipids.

After feeding with high lipid feed for some time, the serum cholesterol and triglyceride levels in rates were raised and an experimental model of rate suffering of from hyperlipernia was obtained. Using this model, hypolipermic drugs were screened. The results of pharmacological activity experiments show that feeding mice with high lipid feed will increase content of serum cholesterol and triglyceride more remarkably than with conventional feed. The compound products can reduce the content of serum cholesterol more remarkably than the high lipid group and can also reduce the content of serum triglyceride.

The original lecithin and cholesterol in the serum were used as substrates, which were kept warm for some time. Because of the effect of lecithin cholesterol acyltransferase (LCAT), cholesterolester (CE) in serum was increased, but free cholesterol (FC) decreased. Then cholesterol was oxidized by cholesterol oxidase and hydrogen peroxide was produced. The latter reacted with 4-amidopyrine and phenol under the action of horseradish peroxidase to produce red quinone imide, which can be used for colorimetric analysis to determine the content of FC. The decrease in FC, ie, the esterification of cholesterol can indicate the activity of LCAT in magnitude. The results of pharmacological activity experiments show that the compound products can enhance the activity of LCAT remarkably whereas the standard control-atromid-S exhibits no remarkable action. This shows that the compound products act better than atromid-S (Table 3).

### BRIEF DESCRIPTION OF THE SEVERAL VIEWS OF THE DRAWING

TABLE 1
Source of raw material and composition of the
five-plant compound of hypolipemic products

| | |
|---|---|
| 1. Rhizoma Alismatis | 25% |
| Alismataceae | |
| Stem tuber of Alisma Orientalis (Sam) Juzep. | |
| 2. Rhizoma Ourcumae Longae | 16.67% |
| Zingberaceae | |
| Stem tuber of Curcumae LongaL. | |
| 3. Herba Gynostemmae | 20.83% |
| Cucurbitaceae | |
| Stem tuber or whole grass of Gynostema Pentaphyllum | |
| (Thunb.) Makino | |
| 4. Fructus Ligustri Lucidi | 20.83% |
| Oleaceae | |
| Fruit of Ligustrum Lucidum Ait. | |
| 5. Herba Epimedii | 16.67% |
| Berberidaceae | |
| Branches and leaves of Epimedium Brevicornum Maxm. | |
| Total | 100% |

TABLE 2
Manufacturing Process and Flowchart for the five-plant Compound Hypolipernic Products

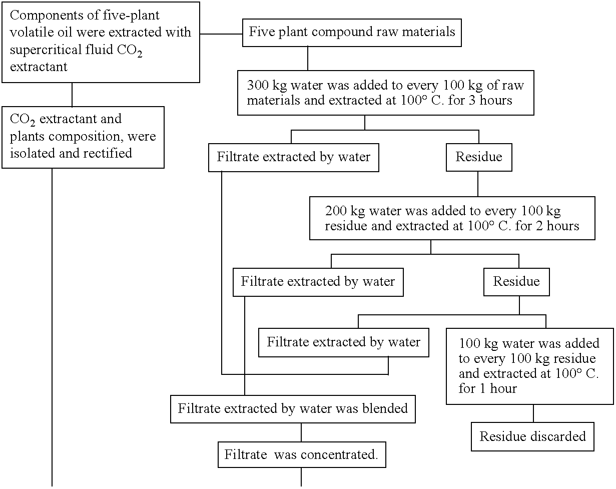

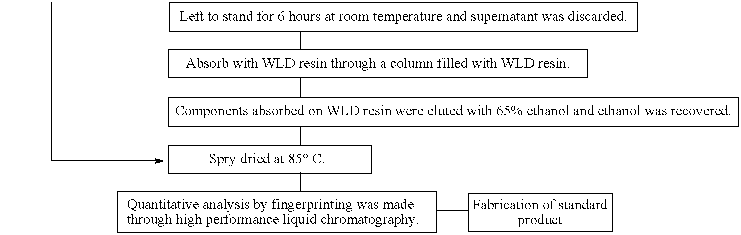

Table 3 Pharmacological Activity of the Five-Plant Compound Hypolipemic Products

1. Effect of the Five-Plant Compound Hypolipemic Products (Briefly Referred to as Compound Products) to Reduce Total Cholesterol in Rabbit Serum

(1) Modelling of hyperlipemia rabbits: white rabbits of 1.8-2.5 kg were chosen regardness of sex and fed with basal feed. After three weeks of adaptation, they were randomly divided into three groups according to weight, each group composed of ten. In addition to basal feed, each group was perfused with 0.2 g/kg cholesterol (with lard as solvent), once a day. The drugs should be administered twice a day. For small dose group, 0.1 mg/kg compound products were perfused; large dose group, 0.3 ml/kg compound products and control group, equal volume of normal saline for 8 consecutive weeks.

(2) At week 2, 4, 6 and 8 after modeling, blood was taken through the ear vein, centrifuged at 3000 rpm and serum was isolated for analysis.

(3) Light density was determined and content of cholesterol calculated.

Result: Compound products can inhibit the rise in serum cholesterol in rabbits fed with cholesterol and high fat feed greatly (Table 1).

TABLE 1
Effect of compound products upon total cholesterol in serum

| | Dose | Number of | Total cholesterol (mg %) (x ± SD) | | | | |
|---|---|---|---|---|---|---|---|
| Group | (ml/kg) | animal (pcs) | Normal value | 2 weeks | 4 weeks | 6 weeks | 8 weeks |
| Control | Normal | 10 | 78.23 ± 12.87 | 374.80 ± 29.86 | 558.62 ± 77.90 | 591.64 ± 60.54 | 692.71 ± 55.88 |
| | saline | | | | | | |
| Compound | 0.1 | 10 | 83.64 ± 17.57 | 331.66 ± 66.94 | 325.82 ± 70.75** | 316.09 ± 54.19 | 303.06 ± 24.25*** |
| products | | | | | | | |
| Compound | 0.3 | 10 | 73.20 ± 10.24 | 302.14 ± 38.50 | 265.19 ± 29.64** | 237.31 ± 20.52*** | 175.64 ± 24.40*** |
| products | | | | | | | |

2. Effect of the Five-Plant Compound Hypolipemic Products Upon the Serum Lipid in Rats Suffering from Experimental Hyperlipemia

Method: Rats of 130±30 g, half male and half female, were randomly divided into three groups. The first group was normal control and fed with conventional feed for fifteen days. Beginning from day 16, 10 ml/kg normal saline was perfused once a day for 4 consecutive weeks. The second group was high lipid control and fed with high lipid feed for fifteen days. Beginning from day 16, 10 ml/kg was perfused once a day for 4 consecutive weeks, the third group was compound products and fed with high lipid feed for fifteen days. Beginning from day 16, 4.2 g crude drug/kg compound products were perfused once a day for 4 consecutive weeks. After the completion of the test, the animals fasted for 8-12 hours, but water was supplied still. Then mixed blood was taken from the artery and vein, collected in the test tube numbered, and centrifuged. A fixed amount of serum was taken and operated as shown in Table 21-3-9.1A and 21-3-9.3A to determine the content of cholesterol and triglyceride respectively.

Result: Compared with mice fed with the normal feed, mice fed with high lipid feed had greater content of serum cholesterol and triglyceride. In comparison with the high lipid group, the compound products group could reduce the content of serum cholesterol greatly and that of serum triglyceride too (Table 2).

TABLE 2
Effect of compound products upon the content
of serum cholesterol and triglyceride in rats
suffering from experimental hyperlipemia (ₓ ± SD) (n = 12)

| Group | Cholesterol (mg %) | Triglyceride (mg %) |
|---|---|---|
| Normal control | 99.6 ± 37.3 | 58.4 ± 23.8 |
| High lipid control | 657.4 ± 207.8ΔΔΔ | 207.8 ± 142.7ΔΔΔ |
| Compound products | 456.1 ± 140.5** | 159.3 ± 63.3** |

3. Effect of the Five-Plant Compound Hypolipemic Products Upon the Activity of Lecithin Cholesterol Acyltransferase in Rats Suffering from Hyperlipemia

(1) 50 wistar rats of 150˜200 g, half male and half female, were randomly divided into five groups, each was composed of ten. One group served as normal control and the remaining four were perused with 17.5 ml/kg cholesterol emulsion once every morning. In the afternoon, one group was given normal saline, the other two were perfused with 3.0 or 6.0 g/kg compound products respectively and another group performed with 0.13 g/kg atromid-s for seven days, after which each group fasted for 12 hours. Blood was taken from the orbit and serum was prepared.

(2) Two portions of 0.1 ml fresh serum were taken from each animal of each group and were preheated in water bath at 37° C. for five minutes, to one portion of which 3 ml cholesterol enzyme-linked reagent application solution was added (30 ml enzyme reaction solution for 1 ml cholesterol enzyme-linked reagent), mixed thoroughly and heated in water bath at 37° C. for 15 minutes while blank control tube and standard tube (0.05 ml standard cholesterol absolute alcohol solution) were made. Zero calibration against bland tube was made by Model 721 spectrophotomer at a wavelength of 500 nm. OD was read and FC content was calculated in terms of Beer's law, which was the serum FC content before reaction. The other portion of serum was kept warm for forty minutes. Then FC content was determined as above, expressed as OD, which was the serum FC content after reaction. LCAT activity (esterification of cholesterol: nmol/ml serum/h) can be calculated by the following formula:

$$LCAT activity = \frac{FC ( OD ) before reaction - FC ( OD ) after reaction}{0.138 \times 386} \times 0.05 \times {10}^{7} \times \frac{2}{3}$$

Result: Compound products can enhance the activity of LCAT greatly at the dose rate of 3.0 g/kg and 6.0 g/kg whereas the action of atromid-s was not noticeable (Table 3)

TABLE 3
Effect of compound products upon the activity of LCAT (ₓ ± SD)

| | | | LCAT activity | |
|---|---|---|---|---|
| | Number | Dose | (TC esterification | |
| Group | of animal (n) | (g/kg) | nmol/ml/h) | P value |
| Normal | 10 | — | 109 ± 7.6 | |
| Compound products | 10 | 3.0 | 628 ± 192 | <0.05 |
| Compound products | 10 | 6.0 | 837 ± 184 | <0.01 |
| Astromid-s | 10 | 0.13 | 536 ± 277 | >0.05 |
| Model | 10 | — | 415 ± 166 | |

### DETAILED DESCRIPTION OF THE INVENTION

1. The pharmacological activity and manufacturing process of the five-plant compound hypolipernic products belong to the combined patent on the pharmacological activity of natural products and methods of isolating and purifying the same. The five-plant compound products are composed of 25% Rhizoma Alismatis, 16.67% Rhizoma Ourcumae Longae, 20.83% Herba Gyostemmae, 20.83% Fructus Ligustri Lucidi and 16.67% Herba Epimedii.

2. The standard products were made by extracting the five-plant raw materials with supercritical fluid CO₂, isolating the gaseous CO₂ and plant components and rectifying the same for future use. Then 300 kg, 200 kg and 100 kg water were added to every 100 kg of the five-plant raw materials, extracted at 100° C. respectively for 3, 2 and 1 hours. The filtrate extracted by water was blended, concentrated at 100° C. and left to stand at room temperature for six hours. The supernatant was discarded. The concentrate was absorbed with WLD resin through a column filled with WLD resin. The components absorbed on WLD resin was eluted with 65% ethanol. After recovery of ethanol, it was blended with the rectified reserve and spray dried at 85° C. Finally a quantitative analysis by fingerprinting was made with high performance liquid chromatography.

3. The pharmacological activity experiment on the effect of the five-plant compound hypolipemic products to reduce total cholesterol in serum of rabbit shows that the compound products can greatly inhibit the rise in serum cholesterol of rabbit fed with cholesterol. The pharmacological activity experiment on the effect upon the activity of lecithin cholesterol acyltransferase in rats suffering from hyperlipemia shows the compound products, compared with the high lipid group, can greatly reduce the serum cholestrole content and also lower serum triglyceride content. Therefore, they are effective against atherosclerosis.

1. Now I declare the pharmacological activity and manufacturing process of the five-plant compound hypolipemic products—Rhizoma Alismatis, Rhizoma Ourcumae Longae, Herba Gynostemmae, Fructus Ligustri Lucidi and Herba Epimedii as my invention.
I think my invention includes the products made by extracting the five-plant compound raw materials with liquid CO₂ first, isolating and rectifying for reserve. Then the five-plant raw materials were extracted with water or ethanol, absorbed with WLD resin, eluted and dried together with the rectified reserve. Finally, a quantitative analysis was made.
I think in my invention, the five-plant compound hypolipemic products have the pharmacological action to inhibit the rise in serum cholesterol in rabbit, enhance LCAT activity and reduce the content of serum cholesterol and triglyceride clinically they can be used in the treatment of atherosclerosis and obesity.
